SEQUENCE LISTING (11 sequences)

SEQ ID NO: 1 - DNA, 25 bp, Artificial Sequence
  HPRT-F synthetic primer
gttggatacaggccagactttgttg

SEQ ID NO: 2 - DNA, 23 bp, Artificial Sequence
  HPRT-R synthetic primer
tactaggcagatggccaggacta

SEQ ID NO: 3 - DNA, 21 bp, Artificial Sequence
  Notchl-F synthetic primer
tgttaatgagtgcatctccaa

SEQ ID NO: 4 - DNA, 23 bp, Artificial Sequence
  Notchl-R synthetic primer
cattcgtagccatcaatcttgtc

SEQ ID NO: 5 - DNA, 22 bp, Artificial Sequence
  Notch2-F synthetic primer
tggaggtaaatgaatgccagag

SEQ ID NO: 6 - DNA, 19 bp, Artificial Sequence
  Notch2-R synthetic primer
tgtagcgattgatgccgtc

SEQ ID NO: 7 - DNA, 18 bp, Artificial Sequence
  Notch3-F synthetic primer
acactgggagttctctgt

SEQ ID NO: 8 - DNA, 18 bp, Artificial Sequence
  Notch3-R synthetic primer
gtctgctggcatgggata

SEQ ID NO: 9 - DNA, 22 bp, Artificial Sequence
  Notch4-F synthetic primer
cacctcctgccataacaccttg

SEQ ID NO: 10 - DNA, 25 bp, Artificial Sequence
  Notch4-R synthetic primer
acacagtcatctgggttcatctcac

SEQ ID NO: 11 - DNA, 19 bp, Artificial Sequence
  Jagged-1 siRNA sequence
ctcgtaatccttaatggtt

(19) United States
(12) Patent Application Publication
(10) Pub. No.: US 2014/0294792 A1
(43) Pub. Date: Oct. 2, 2014

(54) T-REG Cell Expansion

(71) Applicant: The Board of Trustees of the University of Illinois, Urbana, IL (US)

(72) Inventor: Bellur Prabhakar, Oak Brook, IL (US)

(73) Assignee: The Board of Trustees of the University of Illinois, Urbana, IL (US)

(21) Appl. No.: 14/186,766

(22) Filed: Feb. 21, 2014

Related U.S. Application Data
(60) Provisional application No. 61/768,204, filed on Feb. 22, 2013.

(51) Int. Cl. C12N 5/0783; A61K 35/14

(52) U.S. Cl. CPC C12N 5/0637; A61K 35/17
USPC 424/93.71; 435/373; 435/375

(57) ABSTRACT

This invention relates to methods of expanding T regulatory cells through OX40L and Jagged-1 induced signaling. The methods can be used for treating autoimmune diseases.

This application claims priority to U.S. Provisional Patent application 61/768,204 filed Feb. 22, 2013, which is incorporated herein by reference in its entirety.

### STATEMENT OF GOVERNMENT INTEREST

This invention was made with Government support under grant number A1 058190 awarded by the National Institutes of Health. The Government has certain rights in the invention.

### TECHNICAL FIELD

This application relates to the field of immunology. Particularly, this invention relates to methods of expanding T regulatory cells through OX40L and Jagged-1 induced signaling.

### BACKGROUND OF THE INVENTION

T regulatory cells (Tregs) are important cells required for modulation of the immune system, maintaining tolerance to self-antigens and suppression of autoimmune diseases. The emergence of Tregs as a significant component of immune homeostasis provides a potential therapeutic opportunity for active immune regulation and long-term tolerance induction. Indeed, deficiency of naturally occurring T-regulatory cells (nTregs) has been observed in a variety of autoimmune conditions (36, 37). Moreover, adoptive transfer of polyclonal or antigen selected nTregs has been found to overcome autoimmune and allergic conditions (38-40). However, a limitation that prevents therapeutic utilization of Tregs in autoimmune diseases is the relative difficulty in obtaining large numbers of Tregs. Although much is known about T-cell receptor (TCR) mediated T cell activation and proliferation (25), signaling required for Treg proliferation in the absence of TCR stimulation remains largely unknown. Thus, an effective method for expanding Tregs is still needed.

### SUMMARY OF THE INVENTION

This invention provides methods for expanding T regulatory cells through OX40L and Jagged-1 signaling. In accordance with the invention, methods are provided for expanding T regulatory cells comprising co-culturing said T-regulatory cells with one or more of a OX40L⁺ bone marrow derived dendritic cell culture differentiated in the presence of GM-CSF, a Jagged-1⁺ bone marrow derived dendritic cell culture differentiated in the presence of GM-CSF and a OX40L⁺Jagged-1⁺ bone marrow derived dendritic cell culture differentiated in the presence of GM-CSF.

In another aspect, the invention provides methods of treating an autoimmune disease in a patient in need of such treatment comprising administering to the patient a therapeutically effective amount of T-regulatory cells prepared by co-culturing said T-regulatory cells with one or more of a OX40L⁺ bone marrow derived dendritic cell culture differentiated in the presence of GM-CSF, a Jagged-1⁺ bone marrow derived dendritic cell culture differentiated in the presence of GM-CSF and a OX40L⁺Jagged-1⁺ bone marrow derived dendritic cell culture differentiated in the presence of GM-CSF.

In yet another aspect, the invention provides methods for expanding T-regulatory cells comprising co-culturing said T-regulatory cells with one or more of soluble OX40L and soluble Jagged-1. In particular embodiments the OX40L and Jagged-1 are recombinantly produced.

In another aspect, the invention provides methods for treating an autoimmune disease in a patient in need of such treatment comprising administering to the patient a therapeutically effective amount of one or more of soluble OX40L and soluble Jagged-1. In particular embodiments the autoimmune disease is an autoimmune thyroid disease such as Grave's disease or Hashimoto disease. In other embodiments the autoimmune disease is Type 1 Diabetes mellitus. In other embodiments the OX40L and Jagged-1 are recombinantly produced. In yet other embodiments the patient is a human patient.

In another aspect, the invention provides methods treating an autoimmune disease in a patient in need of such treatment comprising administering to the patient a therapeutically effective amount of one or more of OX40L⁺ bone marrow derived dendritic cells differentiated in the presence of GM-CSF, Jagged-1⁻ bone marrow derived dendritic cells differentiated in the presence of GM-CSF and OX40L⁺Jagged-1⁺ bone marrow derived dendritic cells differentiated in the presence of GM-CSF. In particular embodiments the autoimmune disease is an autoimmune thyroid disease such as Grave's disease or Hashimoto disease. In other embodiments the autoimmune disease is Type 1 Diabetes mellitus. In other embodiments the OX40L and Jagged-1 are recombinantly produced. In yet other embodiments the patient is a human patient.

### BRIEF DESCRIPTION OF THE DRAWINGS

FIG. 1. OX40L is necessary but not sufficient for GM-BMDC directed ex vivo expansion of Tregs. (A). Percentage of OX40L⁻ bone marrow derived dendritic cells (GM-BMDCs) differentiated in the presence of granulocyte macrophage colony stimulating factor (GM-CSF) gated on CD11c^{|} cells. (B) GM-BMDCs derived ex vivo from bone marrow cells of WT C57B6/j mice were sorted after 7 days of differentiation with GM-CSF. Naïve carboxy fluorescein succinimidyl ester (CFSE) labelled CD4^{|} T-cells were co-cultured with either splenic dendritic cells (SpDCs), or total, OX40L⁺ or OX40L⁻ enriched GM-BMDCS for 5 days without exogenous antigen and analyzed by FACS. (C). Total, OX40L⁺ or OX40L⁻ GM-BMDCs were co-cultured with Cell-Trace violet labelled sorted GFP+ and GFP− T-cells from Foxp3-GFP mice after CD4⁻ based enrichment for 5 days without exogenous antigen and analyzed by FACS. Co-cultures of GFP⁺ (Foxp3⁺) cells without IL-2 (upper panel) and with IL-2 (lower panel) are shown. (D). Co-cultures of GFP⁻ (Foxp3⁻) cells without IL-2 (upper panel) and with IL-2 (lower panel) are shown. Each scatter plot is representative of five independent experiments, gated over 3500 live CD4⁺ T-cells. Each in vitro experiment was conducted with T-cells, SpDCs and GM-BMDCs pooled from 3 mice. (E) Sorted OX40L⁺ or OX40L⁻ GM-BMDCs were co-cultured with CFSE labelled CD4⁺ T-cells and supplemented with OX40 agonist. Co-cultures were analyzed by FACS on day 5 to determine T-cell proliferation.

FIG. 2. OX40L is necessary but not sufficient in GM-BMDC mediated Treg expansion. (A) CD4⁺ cells from naïve mice were co-cultured with wild type GM-BMDCs either together or in transwells in which the T-cells were exposed to only the BM supernatant; in some cases the T-cells were supplemented with SpDCs and an OX40 agonist. Data were analyzed by FACS. (B) GM-BMDCs from CD80, CD86 and CD80/86 deficient mice were co-cultured with naïve CFSE labelled CD4⁺ T-cells without exogenous antigen and analyzed by FACS (lower panel). Experiments shown in Figures A and B were repeated three times with similar results.

FIG. 3. Jagged-1 mediated Notch signaling is required for Treg expansion by GM-BMDCs. (A) Co-cultures of GM-BMDCs with CFSE labelled CD4⁺ T-cells were supplemented with Gamma-secretase-inhibitor (GSI), an inhibitor of Notch signaling, and analyzed by FACS. (B) Summary of FACS data from Propidium Iodide staining of co-cultures from GSI experiment showing little or no cell necrosis in all co-cultures. (C) Phenotypic characterization of CD11c⁺ SpDCs and GM-BMDCs comparing the levels of expression of different Notch ligands. Cells were gated on the CD11c^{|} populations. (D) Co-cultures of GM-BMDCs with CFSE labelled CD4⁺ T-cells were supplemented with two concentrations of a Jagged-1 neutralizing antibody and analyzed by FACS. Experiments shown in Figures A through D were repeated three times with similar results.

FIG. 4. OX40L and Jagged-1 function are critical for GM-BMDC mediated expansion of Tregs (A) Co-cultures of GM-BMDCs with CFSE labelled CD4^{|} T-cells were supplemented with neutralizing antibodies to Jagged-1 and OX40L, either alone or in combination and analyzed by FACS. (B) FACS analysis of CD25⁺Foxp3⁺ T-cells from the co-cultures of APCs and MHCII^{-/-} GM-BMDCs with CD25⁻ T-cells in the presence and absence of IL-2 and neutralizing antibodies to OX40L and Jagged-1. (C) GM-BMDCs were treated with control or Jagged-1 specific siRNAs. FACS analyses of cell surface expression of Jagged-1 and OX40L showed specific inhibition of Jagged-1, but not OX40L, after Jagged-1 specific siRNA treatment. (D) CFSE labelled CD4⁺ T-cells were cultured with control or Jagged-1 specific siRNA treated GM-BMDC in the presence or absence of anti-OX40L antibodies. Results shown in Figures A through D are representative of 3 independent experiments.

FIG. 5. OX40L/Jagged-1 co-signaling is required for GM-BMDC mediated Treg expansion. (A) GM-BMDCs were analyzed for surface expression of OX40L and Jagged-1. Cells were successively gated over the CD11c⁺ and OX40L⁺ populations and analyzed for Jagged-1 expression. (B) CFSE labelled CD4⁺ T-cells were co-cultured with either total or OX40L^{|} Jagged-1^{|} or OX40L Jagged-1⁻ GM-BMDCs. Some cultures were supplemented with anti-OX40L and/or anti-Jagged-1 antibodies. The Figure shows summary of cell proliferation data analyzed by FACS. The experiment was repeated three times with similar results.

FIG. 6. GM-BMDCs expressing Jagged-1 transduce proliferation signals to Tregs through Notch 3. (A) GFP⁻ and GFP⁻ cells isolated from Foxp3-GFP mice were analyzed for the expression of Notch receptor transcripts by RT-PCR. A Notch 3 transcript was detected specifically in Tregs. cDNAs from different T-cell populations were subjected to PCR using different Notch specific primers and analyzed on 2% agarose gel. Parts of the gel relevant to the specific subpopulation were assembled together. (B) Co-culture of GM-BMDCs and CD4⁺ T-cells in the presence of neutralizing antibody to Notch 3 or Notch 1. Each scatter plot in Figure B and Figure C represents five separate experiments. (C) Shows Notch 3 specific Notch Intracellular Domain (NICD) only in proliferating Foxp3⁺ T-cells in GM-BMDC/T-cell co-cultures analyzed by FACS. CFSE dilution was used to measure cell-proliferation and cells were gated on CFSE diluted or undiluted populations and analyzed for NICD.

FIG. 7. OX40L⁺Jagged-1⁺ GM-BMDCs can induce Tregs in vivo and suppress EAT. (A) Ex vivo expanded Tregs can suppress effector T-cell proliferation. CD4^{|}CD25^{|} T-cells were sorted from the co-culture of OX40L⁺ Jagged-1⁺ GM-BMDCs and T-cells from naive mice. The sorted Tregs were co-cultured with CFSE labelled effector T-cells isolated from ovalbumin (OVA) and mouse thyroglobulin (mTg) immunized mice at different ratios. After 5 days in culture, CD4⁺ T-cells were analyzed for CFSE dilution by FACS. (B) Experimental Autoimmune Thyroiditis (EAT) was induced in mice as described before (1). Briefly, mice were immunized with mTg+CFA on days 1 and 10 to induce EAT. On days 17 and 22, mice were treated with mTg pulsed OX40L⁺ Jagged-1⁺ or OX40L⁺ Jagged-1⁻ GM-BMDCs. Mice were sacrificed on day 35 and analyzed for Foxp3⁺ Tregs in the spleen by FACS. (C) Bar graphs showing percentage of IFN-γ, IL-4 and IL-10 producing CD4⁺ cells in the spleen of treated mice analyzed by FACS. (D) Bar graphs showing percentage of IFN-γ, IL-4 and IL-10 producing CD4⁺ cells in thyroid draining lymph nodes of differently treated mice analyzed by FACS. (E) Hematoxylin and eosin stain (H & E) stained sections of thyroid tissue showing extent of tissue infiltration by lymphocytes. Note no infiltration was detected in unimmunized mice. While significant infiltration is seen in thyroids from mice that were either treated with PBS or with OX40L⁺Jagged-1⁻ GM-BMDCs, there was minimal inflammation in mice treated with OX40L⁺Jagged-1⁺ GM-BMDCs. Results shown are representative of three independent experiments.

FIG. 8. Inhibition of Notch signaling abrogates GM-BMDC mediated Treg proliferation. Co-cultures of BMDCs with Cell Trace-violet labelled CD4⁺ T-cells were supplemented with R04929097, a Gamma-secretase-inhibitor (GSI), in different concentrations and analyzed by FACS. The inhibition of the Notch signaling by the GSI resulted in abrogated GM-BMDC mediated Treg proliferation.

FIG. 9. Treatment of NOD mice with soluble OX40L/Jagged-1 leads to increased percentage of Foxp3 Tregs in the spleen and lymph nodes. 10-week old NOD mice were treated 3-times with PBS or soluble recombinant OX40L (200 μg/dose) and soluble recombinant Jagged-1 (100 μg/dose). Spleen and lymph node tissues were analyzed. Mice receiving OX40L & Jagged-1 showed a significant increase in the percentage of Foxp3⁺ Tregs in the spleen (i.e, 20.1% in PBS treated vs 32.3% in ligand treated), pancreatic (15.5 vs 26.6%) and peripheral lymph nodes (11.3% vs 28.1%), indicating that soluble OX40L & Jagged-1 treatment can cause Treg expansion in-vivo.

FIG. 10. Treatment of NOD mice with soluble OX40L/Jagged-1 did not affect percentages of T-cells or B-cells in lymphoid organs. 10-week old NOD mice were treated 3-times with PBS or soluble recombinant OX40L (200 μg/dose) and soluble recombinant Jagged-1 (100 μg/dose). Spleen and lymph node tissues were analyzed. Treatment did not affect the percentages of CD4⁺, CD8⁺ and B220⁺ cells in the lymphoid organs.

FIG. 11. OX40L/Jagged-1 treatment does not impair normal physiological functions of liver and kidney. 10-week old NOD mice were treated 3-times with PBS or soluble recombinant OX40L (200 μg/dose) and soluble recombinant Jagged-1 (100 μg/dose). Serum calcium and BUN tests were used as indicators or normal renal function. Serum alkaline phosphatase, total bilirubin total protein and albumin were used as indicators of normal liver function.

FIG. 12. H&E stained pancreatic tissue sections showed no B-cell damage upon OX40/Jagged-1 treatment. 10-week old NOD mice were treated 3-times with PBS or soluble recombinant OX40L (200 μg/dose) and soluble recombinant Jagged-1 (100 μg/dose). Pancreatic tissue sections were stained with H&E. The stained pancreatic tissue did not show any cell damage following OX40L and Jagged-1 treatment (OX40L/J-1).

### DETAILED DESCRIPTION OF THE INVENTION

The invention provides methods for expanding T-regulatory cells (Tregs) using OX40L and Jagged-1 induced signaling. In particular embodiments, the OX40L and/or Jagged-1 are expressed on bone marrow derived dendritic cells differentiated in the presence of GM-CSF (GM-BMDC). Additionally, OX40L and Jagged-1 can be used in the soluble form for expansion of Tregs. The invention also provides methods for treating autoimmune diseases by increasing the number of Tregs as a result of OX40L and Jagged-1 induced signaling.

Unless otherwise explained, all technical and scientific terms used herein have the same meaning as commonly understood by one of ordinary skill in the art to which a disclosed disclosure belongs.

The singular terms “a,” “an,” and “the” include plural referents unless context clearly indicates otherwise. Similarly, the word “or” is intended to include “and” unless the context clearly indicates otherwise.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms “T regulatory cell” or “Tregs” as used herein refer to a cell that can modulate a T cell response. Tregs express the transcription factor Foxp3, which is not upregulated upon T cell activation and discriminates Tregs from activated effector cells. Tregs are classified into natural or adaptive (induced) Tregs on the basis of their origin. Foxp3⁺ natural Tregs (nTregs) are generated in the thymus through MHC class II dependent T cell receptor. Adaptive Tregs are non-regulatory CD4+ T-cells which acquire CD25 (IL-2R alpha) expression outside of the thymus, and are typically induced by inflammation and disease processes, such as autoimmunity and cancer. The methods described herein can employ Tregs that expresses one or more of CD4, CD25 and Foxp3.

OX40L belongs to the tumor necrosis factor superfamily with co-stimulatory function. OX40L when expressed on antigen-presenting cells binds to OX40 expressed on T-cells.

The Jagged members (Jagged-1 and Jagged-2) of Notch family ligands have been shown to play important role in Treg expansion (12, 13). The Notch family has 4 known receptors, Notch-1, -2, -3 and -4, and five known Notch ligands namely, DLL1, DLL3 and DLL4, and Jagged-1 and Jagged-2. Upon ligand binding, Notch receptors undergo two proteolytic cleavages. The first cleavage is catalysed by ADAM-family metalloproteases and is followed by the gamma-secretase mediated release of Notch intracellular domain (NICD). The NICD translocates to the nucleus where it forms a heterodimeric complex with various co-activator molecules and acts as a transcriptional activator (15). Expression of specific Notch ligands on dendritic cells (DCs) is known to activate specific T-cell responses (14). While Jagged ligands have been shown to direct naive T-cells toward Th2 and/or Treg type of responses, Delta like ligands (DLL) have been shown to skew them towards a Th1 response (16). Of relevance to the current invention are earlier reports of Treg expansion by hematopoietic progenitors expressing Jagged-2 and APCs over-expressing Jagged-1 (12, 13, 17, 18). Similarly, DLL4 blockade ameliorated experimental autoimmune encephalomyelitis (EAE) (20).

While OX40 is constitutively expressed on Tregs (27), Notch 3 is preferentially expressed on Tregs (24). In the context of TCR signaling, OX40 mediated-signaling can increase T cell proliferation by activating PI3 kinase (PI3K) and Akt, which are upstream activators of mTOR (28). GM-BMDCs derived from MHC class-II knockout mice were also able to expand Tregs and indicated that TCR signaling was not necessary (8). OX40 activation can form a signalosome consisting of CARMA1, PKC-Q and TRAF2 and cause enhanced NF-KB activation and contribute to cell survival and expansion (29, 30). Notch 3 has been reported to activate both the alternate and the canonical NF-KB pathways. It can activate the alternative (RelB) NF-KB pathway in murine thymocytes (31) via cytoplasmic IKKα and cooperate with canonical NF-KB in stimulating FoxP3 expression (32). Thus NF-KB may be an important point of convergence between OX40 and Notch 3 signaling in Tregs.

Notch 1 has been reported to maintain expression of FoxP3 in peripheral Tregs in collaboration with TGFβ (33). Therefore, it is possible that different Notch paralogs can maintain FoxP3 expression depending on other signals and cellular context. It is well known that Foxp3⁺ Tregs are unable to proliferate or proliferate poorly when stimulated (34, 35) and upon proliferation they lose Foxp3 expression. Notch 3 has been shown to co-operatively regulate Foxp3 expression through trans-activation of the Foxp3 promoter (32). Therefore, it is likely that the interaction of Jagged-1 with Notch 3 helps sustain Foxp3 transcription while OX40 signalosome formation, in the absence of TCR signaling, may drive Foxp3⁺ Treg cell-proliferation. Thus, concurrent signals from Notch 3 and OX40 can allow Treg proliferation while sustaining Foxp3 expression.

Using GM-BMDC from MHC class-II deficient mice, OX40L mediated ex vivo expansion of Tregs has been shown not to require T-cell receptor (TCR) stimulation per se although it was critically depended on exogenous IL-2 (8). Notch 3 mediated signaling has been reported to sustain regulatory phenotype on Tregs (26). Furthermore, thymocytes and T cells from transgenic mice expressing Notch 3 NICD (N3-tg mice) in which Notch3 is constitutively active contain a significantly higher proportion of CD4⁺CD25⁺ cells (24).

In particular aspects of the invention, the method includes expanding T-regulatory cells by co-culturing T-regulatory cells with a bone marrow-derived dendritic cell differentiated in the presence of GM-CSF that expresses a co-stimulatory molecule such as OX-40L and/or Jagged-1. The term “expanding” as used herein refers to increasing the number of cells in the cell population due to cell replication.

Treatment with low dose GM-CSF has been found to be sufficient to prevent the development of Experimental Autoimmune Thyroiditis (EAT) in CBA mice, Experimental Autoimmune Myasthenia Gravis (EAMG) in C57BL mice and Type 1 Diabetes (T1D) in NOD mice (1-3). Moreover, such treatment reversed ongoing EAT and EAMG, and restored normal thyroid and neuromuscular conduction respectively (1, 2). Others have shown similar protective effect of GM-CSF in T1D and Irritable Bowel Disease (IBD) (4, 5). Additionally, the therapeutic effect of GM-CSF was primarily mediated through the mobilization of CD11c⁺CD8α⁻DCs (6), which caused expansion of regulatory Tregs. These expanded Tregs suppressed the disease through increased IL-10 production (7).

Additionally, GM-CSF can differentiate bone marrow derived DC precursors ex vivo and cause a selective expansion of CD11c⁺CD11b⁺CD8α⁻CDCs (GM-BMDCs) (8). Unlike DCs isolated from the spleen (SpDCs), these ex vivo developed GM-BMDCs were able to directly and specifically expand Tregs upon co-culture with CD4⁺ T-cells. Furthermore, treatment of mice with GM-CSF led to an increase in CD11c⁺CD11b⁺CD8α⁻DCs in vivo with concomitant increase in Foxp3⁺ Tregs, suggesting a parallel mechanism of CD11c⁺CD11b⁺CD8α⁻DCs mediated Treg expansion ex vivo and in vivo.

Using GM-BMDCs from MHC class-II^{-/-} mice, it has been shown that Treg expansion by these DCs did not require canonical antigen presentation to TCR but required exogenous IL-2 (8). Using blocking antibodies to co-stimulatory molecules expressed on the surface of GM-BMDCs, it was shown that the GM-BMDC mediated Treg proliferation was dependent upon GM-BMDC bound OX40L (8), a member of the tumor necrosis factor super family with co-stimulatory function (9). Studies by other groups have also suggested a novel role for OX40L-OX40 interaction in the expansion of Tregs (10, 11).

In other aspects of the invention, the methods include expanding T-regulatory cells by co-culturing the T-regulatory cells with soluble OX40L and/or soluble Jagged-1.

The term “soluble” as used herein describes molecules that lack any transmembrane domain or protein domain that anchors or integrates the polypeptide into the membrane of a cell expressing such polypeptide.

In other aspects of the invention, the methods include treating an autoimmune disease using the Tregs produced through OX40L and/or Jagged-1 induced signaling. For example, an autoimmune disease in a patient in need of such treatment can be treated using the Tregs produced as a result of co-culturing Tregs with a therapeutically effective amount of one or more of a OX40L⁺ GM-BMDC, a Jagged-1⁺ GM-BMDC and a OX40L⁺Jagged-1⁺ GM-BMDC. Additionally, the Tregs produced by any of the methods disclosed herein can be used for treatment of an autoimmune disease in a patient in need thereof.

In other particular aspects, the method includes treating an autoimmune disease in a patient in need of such treatment comprising administering to the patient a therapeutically effective amount of one or more of soluble OX40L and soluble Jagged-1.

The term “patient” as used herein refers to a mammal suffering from an autoimmune disease. In certain particular embodiments, the mammal is a human. In other certain embodiments, a patient is a human suffering from an autoimmune disease.

The term “autoimmune diseases” as used herein refers to a disease resulting from an immune response against a self-tissue or tissue component, including both self-antibody responses and cell-mediated responses. Exemplary autoimmune diseases that are suitable as targets for the inventive methods are type I diabetes mellitus (T1D), Crohn's disease, ulcerative colitis, myasthenia gravis, vitiligo, Graves' disease, Hashimoto's disease, Addison's disease and autoimmune gastritis and autoimmune hepatitis, rheumatoid disease, systemic lupus erythematosus, progressive systemic sclerosis and variants, polymyositis and dermatomyositis, pernicious anemia including some of autoimmune gastritis, primary biliary cirrhosis, autoimmune thrombocytopenia, Sjogren's syndrome, multiple sclerosis and psoriasis. One skilled in the art understands that the methods of the invention can be applied to these or other autoimmune diseases, as desired.

As used herein, the term “amount effective,” “effective amount” or a “therapeutically effective amount” refers to an amount of compound or composition sufficient to achieve the stated desired result, for example, treating or limiting development of autoimmune disease. The amount of the compound or composition which constitutes an “effective amount” or “therapeutically effective amount” may vary depending on the severity of the disease, the condition, weight, gender or age of the patient to be treated, the frequency of dosing, or the route of administration, but can be determined routinely by one of ordinary skill in the art. A clinician may titer the dosage or route of administration to obtain the optimal therapeutic effect.

In particular embodiments the autoimmune disease is an autoimmune thyroid disease (e.g., Grave's disease and Hashimoto disease). Autoimmune thyroid disease involves the dysfunction of the diseased thyroid gland and varies from hypothyroidism due to glandular destruction in Hashimoto's thyroiditis or blocking antibodies in primary myxedema to hyperthyroidism in Graves' disease due to thyroid simulating antibodies. In other particular aspects the autoimmune disease is Type 1 Diabetes Mellitus.

Cellular therapies for autoimmune diseases, including formulations and methods of administration are known in the art and can be applied to the T-regulatory cells and vectors described herein. See, for example, in EP1153131 A2, incorporated herein by reference.

A polypeptide of the invention can be produced recombinantly. A polynucleotide encoding a polypeptide of the invention can be introduced into a recombinant expression vector, which can be expressed in a suitable expression host cell system using techniques well known in the art. A variety of bacterial, yeast, plant, mammalian, and insect expression systems are available in the art and any such expression system can be used.

The foregoing may be better understood by reference to the following examples which are presented for purposes of illustration and are not intended to limit the scope of the invention.

### EXAMPLES

Materials and Methods:

Animals: Six to eight week old CBA/J mice were purchased from the Jackson Laboratory. Mice were housed and provided food and water ad libitum. CD80−/−, CD86−/−, CD80−/− CD86−/− Foxp3GFP and WT (C57B6/j background) mice were kindly provided by Dr. Chenthamarakshan Vasu (Department of Surgery, Medical University of South Carolina).

GM-CSF, antibodies and thyroglobulin: Recombinant GM-CSF and CFSE were purchased from Invitrogen (Carlsbad, USA). Phycoerythrin-conjugated anti-H-2K^{d} (MHC class II), anti-Jagged-1, anti-DLL1, anti-DLL3, anti-DLL4, anti-Notch 1; Pacific blue conjugated anti-CD4, APC conjugated anti-CD11c, anti-CD11b, anti-Foxp3, antiCD3, PE conjugated anti-IL-4, and IFN-γ antibodies, and OX40 agonist (OX86) were purchased from eBioscience (San Diego, Calif.). APC conjugated anti-OX40L antibody was purchased from Biolegend (San Diego, Calif.). Blocking antibodies to OX40L (AF1236), Jagged-1 (AF599), Notch 1 (AF1057) and Notch 3 (AF1308) and normal goat IgG control (AB-108-C) were purchased from R&D systems (Minneapolis, Minn.). Primary and secondary antibodies for staining intracellular Notch receptors (NICD) against Notch 1 (sc-23307) and Notch 3 (se-5593) (12, 21) were purchased from Santa Cruz Biotechnology (Santa Cruz, Calif.). Mouse thyroids were purchased from Pel-Freeze (Rogers, Ariz.) and thyroglobulin was prepared as described earlier (22). In brief, thyroids were homogenized in 2.5 ml PBS with pestle-homogenizer (Wheaton, Millville, N.J.) with overnight extraction at 4° C. The extract was clarified by centrifugation (15000×g) and fractionated on a Sephadex G-200 column (2.5 cm×90 cm) that had been equilibrated with 0.1 M phosphate buffer, pH 7.2. The concentration and purity of mTg was determined spectrophotometrically at 280 nm and by resolving on 7% SDS-PAGE followed by Coomassie blue staining. Gamma-secretase inhibitors (GSI) S-2188 and R04929097 were purchased from Sigma-Aldrich (St. Louis, Mo.) and Selleck Chemicals (Houston, Tex.) respectively.

Isolation of DC and T-cell subpopulations: Bone marrow cells were cultured in complete RPMI medium containing 10% heat-inactivated FBS in the presence of 20 ng/ml GM-CSF for 3 days. On days 4 and 6, fresh medium containing 20 ng/ml GM-CSF was added. Non-adherent CD11c⁺ DCs from eight day old cultures were enriched using anti-CD11c coated magnetic beads according to the manufacturer's directions (Miltenyi Biotech, Auburn, Calif.). Specific sub-populations of GM-BMDCs and CD4+CD25+ T-cells were sorted using a MoFlo flow cytometer (Beckman/Coulter, Ranch Cucamonga, Calif.) following staining with appropriate antibodies (OX40L, Jagged-1, CD4, CD25). To obtain GFP+ and GFP− cells, total CD4+ cells were first separated using CD4 microbeads (Miltenyi Biotech) and then the cells were sorted based on GFP expression using a MoFlo flow cytometer (Beckman/Coulter).

In vitro co-cultures of DCs and T cells: Each in vitro experiment was conducted in triplicate with T-cells, SpDCs and GM-BMDCs pooled from 3 mice. GM-BMDCs (5×10⁴) and CD11c⁺ SpDCs were cultured with CD4⁺, CD4⁺CD25⁻ and CD4⁺CD25⁺ T-cells at a ratio of 1:2 for 5 days. For proliferation assays, T-cell subpopulations were labelled with CFSE at 10 μM according to manufacturer's instruction (Invitrogen, Carlsbad, Calif.) before co-culturing them with DCs. Some cultures were supplemented with IL-2 (10 U/ml) (R&D Systems), antiOX40L (up to 10 μg/ml) antibody, OX40 agonist (OX-86, 5-10 μg/ml), anti-Jagged-1 (10-20 μg/ml) antibody or anti-Notch3 (10-20 μg/ml) antibody. For blocking experiments with anti-OX40L or anti-Jagged-1 antibodies, GM-BMDCs were pre-treated with the indicated antibodies for 30 min at 37° C. and then used in co-culture with naive CD4⁺ T-cells. For blocking experiments with anti-Notch3 antibody, CD4+ T-cells isolated from mouse splenocytes were first treated with anti-Notch3 antibody at two different concentrations (10 and 20 μg/ml) or with 20 μg/ml of an anti-Notch 1 antibody, incubated at 37° C. for 30 minutes and then co-cultured with GM-BMDCs/SpDCs for 5 days. Some co-cultures were supplemented with different concentrations of gamma-secretase inhibitors (GSI) S-2188 (5 and 10 μM) or R04929097 (200 nM-5 μM).

Suppression assay: CD4⁺CD25⁻ effector T-cells were isolated from spleens, stained with CFSE and plated into flat bottom 96 well plates at 0.5×10⁶ cells/well in the presence of either OVA or mTg (100 μg/ml) and splenic antigen presenting cells (APCs). Sorted CD4⁺CD25⁺ Tregs from ex vivo co-cultures of naïve CD4⁺ T-cells and GM-BMDC were added at different ratios to the co-culture containing CD4⁺CD25⁻ T-cells from primed mice.

Propidium iodide (PI) and Intracellular Staining: Briefly, at the end of co-culture experiments, T-cells were stained with Pacific blue labelled anti-mouse CD4 antibody and cells labelled with propidium iodide and subjected to FACS analysis to assess cell viability. For intracellular staining, surface stained cells were fixed and permeabilized using a commercial kit and according to the manufacturer's instructions (eBioscience) and incubated with specified antibodies.

FACS: Freshly isolated and ex vivo cultured cells were washed with PBS-BSA-EDTA. For surface staining, the cells were labelled with specified FITC, PE, APC conjugated antibodies for 30 min. For cell proliferation assays, the cells were labelled with CFSE, fixed, permeabilized and incubated with fluorescent coupled antibodies for intracellular staining. Stained cells were washed three times and analyzed by Cyan flow cytometer (Beckman/Coulter).

siRNA transfection into GM-BMDC: A 21 bp siRNA sequence (Dharmacon, Lafayette, Colo.) specific to Jagged-1 (5′-CTCGTAATCCTTAATGGTT-3; SEQ ID NO: 11) was used at a final concentration of 120 nM as previously described (23). Briefly, 3 μl of 20 μM annealed siRNA was incubated with 3 μl of GenePorter (Gene Therapy Systems) in a volume of 94 μl of serum-free RPMI 1640 at room temperature for 30 min. This mixture was added to each well containing GM-BMDC in a volume of 500 μl and incubated for 4 h at 37° C. 3 μl of GenePorter alone was used for mock transfection as a negative control. After incubation, 500 μl/well of RPMI 1640 supplemented with 20% FCS was added and twenty-four hours later, GM-BMDCs were washed and used.

RT-PCR: Total RNA was extracted using TRIzol reagent (Invitrogen) and the first strand cDNA was synthesized using Superscript 2 (Invitrogen). Gene specific primers were used for semi quantitative PCR amplification (0.5 min at 94° C., 0.5 min at 55° C., and 0.5 min at 72° C. for 33 cycles) to detect relative amounts of different transcripts. The following primer sets were used to amplify the indicated products:

SEQ ID NO: 1
HPRT-F, GTTGGATACAGGCCAGACTTT′GTTG
SEQ ID NO: 2
HPRT-R, TACTAGGCAGATGGCCAGGACTA
SEQ ID NO: 3
Notch1-F, TGTTAATGAGTGCATCTCCAA
SEQ ID NO: 4
Notch1-R, CATTCGTAGCCATCAATCTTGTC
SEQ ID NO: 5
Notch2-F, TGGAGGTAAATGAATGCCAGAG
SEQ ID NO: 6
Notch2-R, TGTAGCGATTGATGCCGTC
SEQ ID NO: 7
Notch3-F, ACACTGGGAGTTCTCTGT
SEQ ID NO: 8
Notch3-R, GTCTGCTGGCATGGGATA
SEQ ID NO: 9
Notch4-F, CACCTCCTGCCATAACACCTTG
SEQ ID NO: 10
Notch4-R, ACACAGTCATCTGGGTTCATCTCAC

Priming mice with mTg and OVA: Groups of CBA/J mice were immunized (3 mice per group for each experiment) subcutaneously with OVA (100 μg/mouse) or mTg (100 μg/mouse) emulsified in Complete Freund's Adjuvant (CFA) on day 1 and day 10. Various subsets of T cells from these mice were used in Treg expansion and proliferation assays.

Adoptive transfer: Three groups of 3 mice each were immunized twice, 10 days apart, with mTg (100 μg/ml) emulsified in CFA. Ten days after the 2^{nd} immunization, mice received i.v. injection of either i) PBS, ii) 2×10⁶ purified CD11c⁺ DCs from untreated CBA/J mice or iii) 2×10⁶ CD11c⁻ GM-BMDC purified and sorted from BM cultures. Two identical adoptive transfers were done for each group at 5 day intervals. Five-days after the 2^{nd} transfer, mice were sacrificed and spleen and thyroid draining lymph node cells were analyzed for Treg percentages.

Statistical analysis: Mean, standard deviation, and statistical significance were calculated using the Graph pad software and MS-Excel application software. Statistical significance was determined using the one tailed Students t-test. A p value of ≦0.05 was considered significant.

### Example 1

### OX40L is Necessary but not Sufficient for the Expansion of Tregs Mediated by GM-BMDC

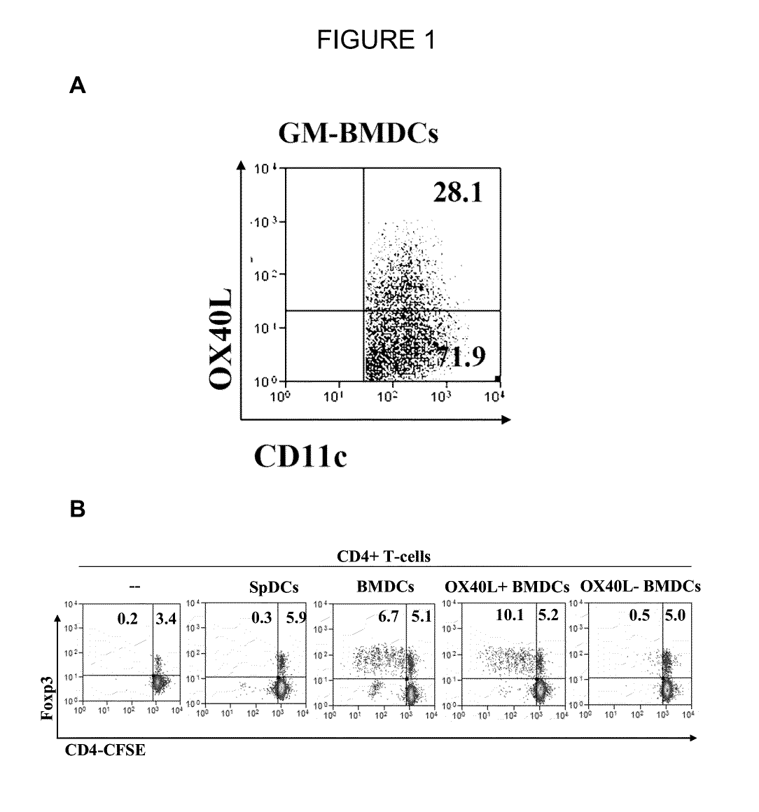

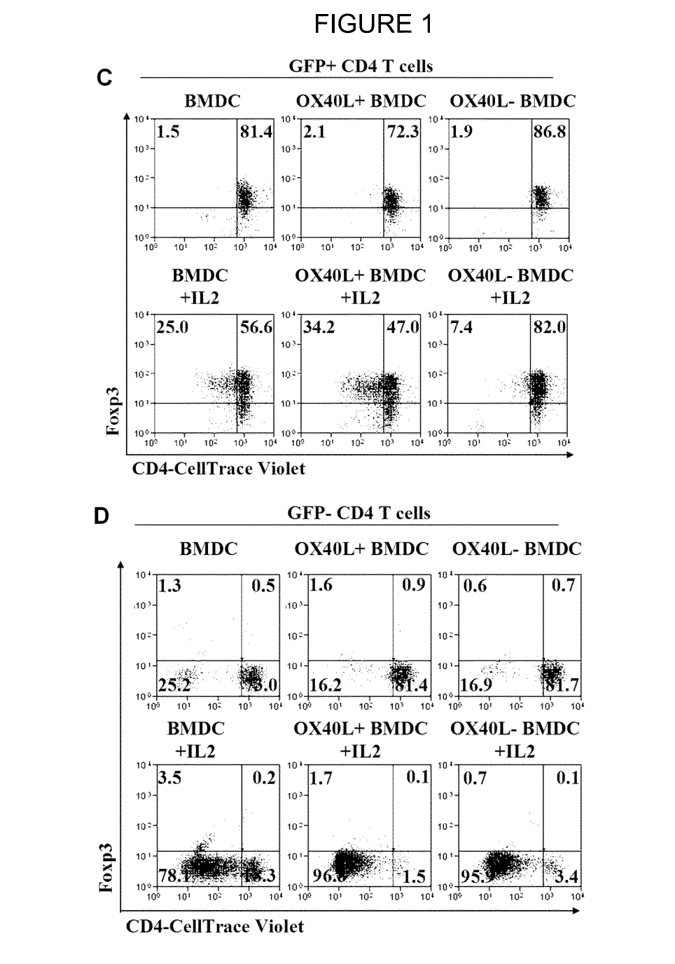

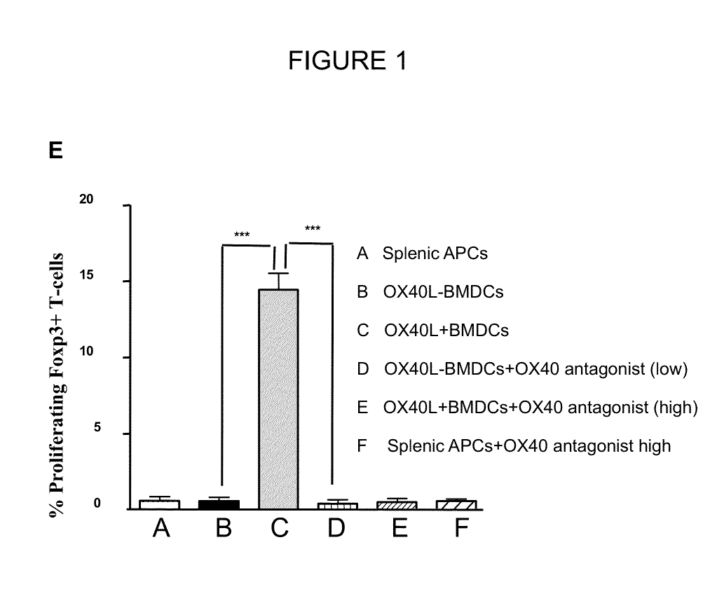

A blocking antibody against OX40L demonstrated a dose-dependent abrogation of Treg proliferation by GM-BMDC (8), which was restored upon addition of a soluble OX40 agonist. In a typical 7-day bone marrow culture with GM-CSF, ˜30% CD11c⁺ GM-BMDCs were OX40L⁺ (FIG. 1A). To determine if OX40L-induced signaling was sufficient for the expansion of Tregs, co-cultures with sorted populations of OX40L⁺ and OX40L⁻ GM-BMDCs with naive CD4⁺ T-cells were established. Only OX40L^{|} GM-BMDC drove the proliferation of Foxp3^{|} Tregs (10.1±0.6%) relative to OX40L⁻ GM-BMDC (0.5±0.1%, p=0.002) (FIG. 1B).

To specifically address the role of OX40L⁺ GM-BMDCs on Foxp3⁻ Tregs, Foxp3-GFP transgenic mice were used. Co-cultures of sorted OX40L^{|} and OX40L⁻ GM-BMDCs (FIG. 1) with sorted and Cell-Trace Violet labelled CD4⁺GFP⁺ (FIG. 1C) or CD4⁻ GFP⁻ (FIG. 1D) T cells isolated from Foxp3-GFP mice (FIG. 1), in the presence or absence of IL-2 were established. The extent of Cell-Trace Violet dilution revealed that in the absence of IL-2, a very small fraction of GFP⁺ T-cells proliferated after 5-days of co-culture with either total, OX40L⁺ or OX40L− GM-BMDCs. However, in the presence of IL-2, Foxp3+ T-cells proliferated efficiently only when co-cultured with either total (25.0±1.7%) or OX40L⁺ (34±3.2%), and not with OX40L⁻, GM-BMDCs (7.4±1.0%). In contrast, GFP⁻ T-cells (Foxp3⁻) showed either modest or robust proliferation based on absence or presence of IL-2 irrespective of whether they were co-cultured in the presence of total, OX40L⁺ or OX40L⁻ GM-BMDCs. Most notably, there was not any adaptive conversion of Teff into Tregs in any cultures involving GFP− cells. It is important to note that none of these co-cultures were stimulated with anti-CD3 or any exogenous antigen. Thus, the data strongly suggested that only OX40L⁺ GM-BMDCs can cause efficient proliferation Foxp3+ Tregs.

To determine if signaling by OX40L alone was sufficient to expand Tregs, CD4 T cells co-cultured with either OX40L⁻ GM-BMDC or splenic DCs were supplemented with a functional OX40 agonist. Such a treatment failed to cause significant proliferation of Foxp3⁺ Tregs (0.8±0.1%) when compared to the Treg proliferation noted in the presence of OX40L⁺ GM-BMDC (13.5±0.7%, p<0.001) (FIG. 1E). These results suggested that OX40L, although required, may not be sufficient for the GM-BMDC mediated ex vivo expansion of Tregs.

### Example 2

### Surface Bound Ligands Other than the B7 Family Co-Stimulatory Molecules are Involved in GM-BMDC Induced Tree Expansion

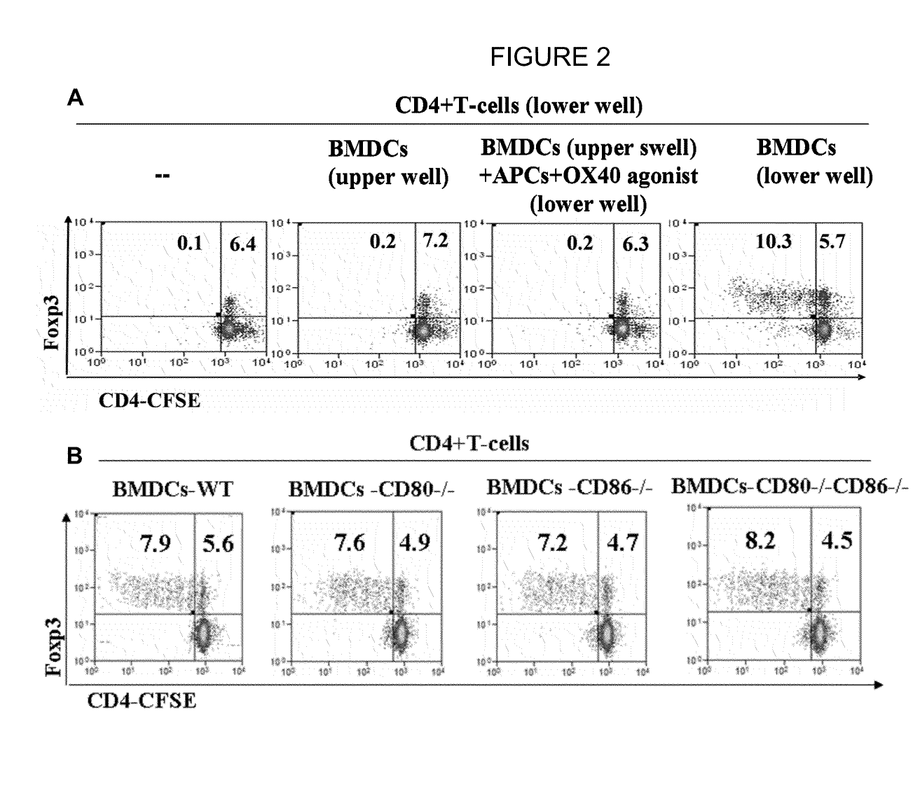

Co-cultures of CD4⁻ T-cells and DCs in trans-well plates were established to determine if, in addition to OX40L expressed on GM-BMDC, co-signaling by a soluble factor from, or a surface bound molecule on, GM-BMDC is required for Treg expansion. Splenic APCs and CD4⁻ T-cells along with an OX40 agonist were physically separated from GM-BMDC cultured in trans-wells, which allowed for free exchange of soluble factors in culture medium. If soluble factors from GM-BMDC were contributing to Treg expansion, those factors would be expected to cross the trans-well barrier and aid in Treg expansion in the presence of OX40 agonist and splenic APCs. However, there was little or no proliferation of Tregs (0.2±0.1%) in the trans-well when compared to CD4+T-cell-GM-BMDC co-cultures (10.3±0.7%) (FIG. 2A). These results suggested that in addition to OX40L, co-signaling by other GM-BMDC surface bound molecule(s) was essential for GM-BMDC mediated Treg expansion.

To identify other cell surface molecule(s) involved in GM-BMDC mediated Treg proliferation, naive CD4+ T-cells were co-cultured with GM-BMDC derived from CD80 and CD86 knockout mice. Lack of expression of either CD80 or CD86 on GM-BMDC had little or no effect on their ability to induce Treg proliferation (7.6±1.0% and 7.2±0.8%) relative to GM-BMDC derived from WT mice (7.9±0.6%) (FIG. 2B). In fact, GM-BMDC developed ex vivo from CD80/CD86 double knock-out mice could cause robust Treg proliferation in co-cultures (8.1±0.9%). These data strongly suggested that a molecule(s) other than CD80 or CD86 was involved in signaling required for the GM-BMDC induced Treg expansion.

### Example 3

### Jagged-1 Mediated Notch Signaling is Involved in GM-BMDC Induced Proliferation of Foxp3⁺ Tregs

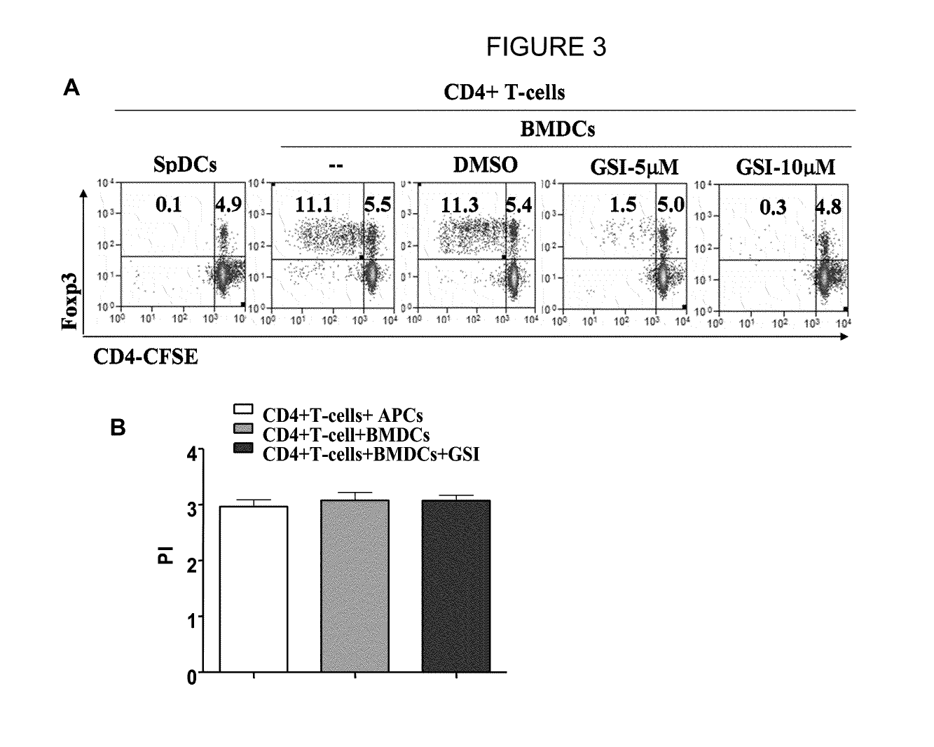

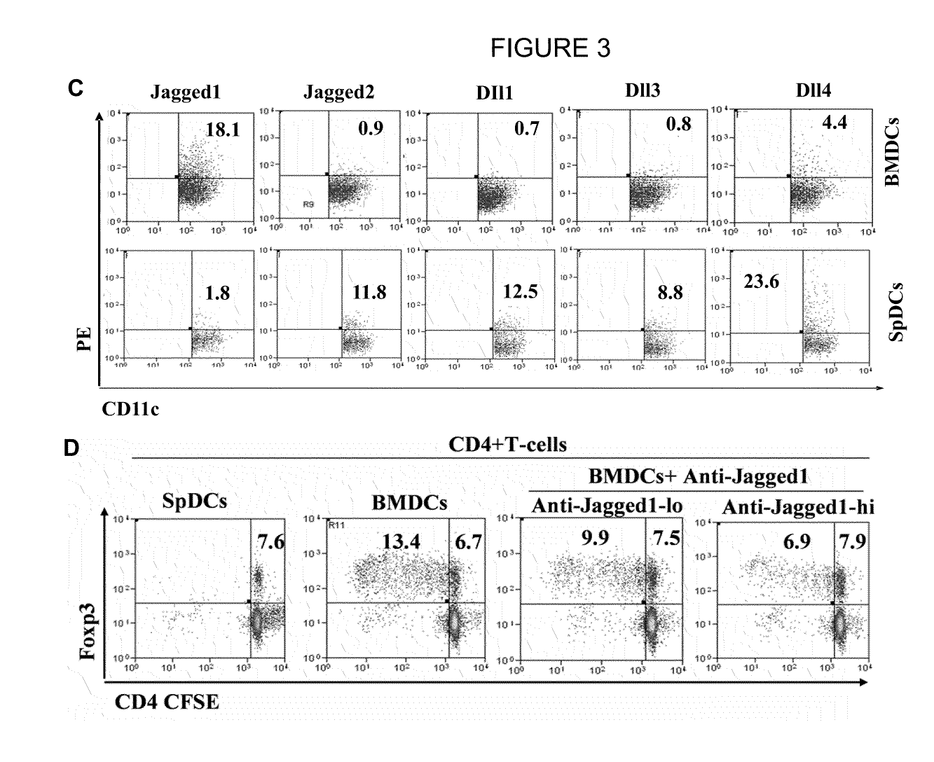

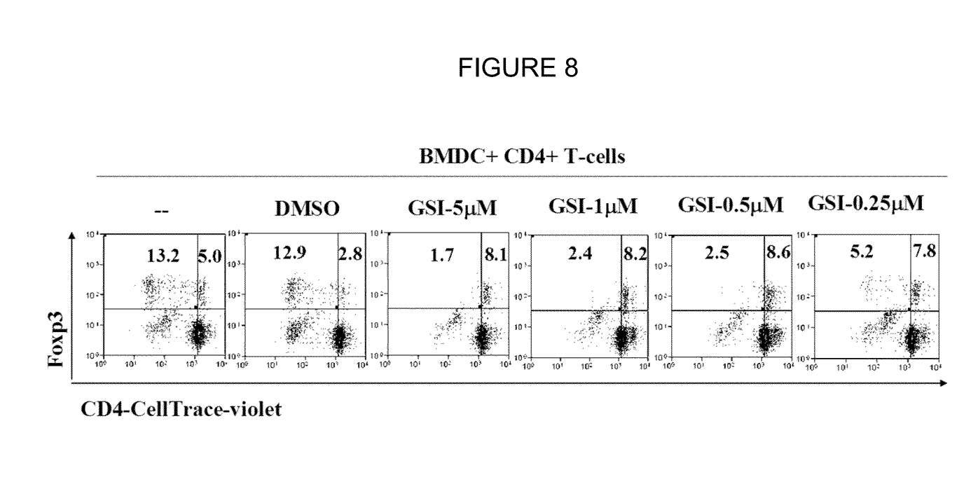

To test whether Notch signaling was involved in ex vivo Treg proliferation, S-2188, a γ-secretase inhibitor (GSI) that blocks Notch signaling was added to the GM-BMDC/T-cell co-cultures. Blocking Notch signaling with S-2188 completely abrogated Treg proliferation (1.5±0.3%-0.3±0.1%) in a dose dependent manner (5-10 μM) compared to proliferation of Tregs in untreated cultures (11.1±1.0%, p<0.001) (FIG. 3A). To assess whether this difference was attributable to a difference in cell viability, co-cultures were stained with propidium iodide (PI) and analyzed by FACS for cell death; S-2188 treatment did not affect cell survival (FIG. 3B). The effect of treating the cells with R04929097, another GSI known to be effective at lower doses, at different concentrations (250 nM-5 μM) (FIG. 8) was also tested. While co-cultures of CD4+ T-cells with GM-BMDCs alone resulted in robust proliferation (˜13.2±0.4%), treatment with GSI severely restricted proliferation in a dose dependent manner (e.g., 1.7±0.3% at 5 μM; and 5.2±0.4% at 250 nM GS1). These results suggested that Notch signaling was important for GM-BMDC mediated Treg proliferation. Subsequently, GM-BMDC and SpDCs were stained to analyze for the expression of different Notch ligands. A much higher proportion of GM-BMDC expressed Jagged-1 (18.1±2.8%, p<0.01) relative to SpDCs (1.8±0.5%) (FIG. 3C). In contrast, all other Notch ligands (Jagged-2, DLL1, DLL3 and DLL4) were expressed on a higher percentage of SpDCs than on GM-BMDC. Addition of a blocking antibody against Jagged-1 (lo=10 μg/ml; hi=20 μg/ml) suppressed Treg expansion in a dose dependent manner (reduced from 13.4%±1% to 9.9%±0.5% with low dose and to 6.9±0.2% with high dose (p<0.01 in all instances) (FIG. 3D). Jagged-1 blocking antibody had little or no effect on the percentages of non-dividing Tregs (˜7-8%) and indicated that the effect of Jagged-1 blockage primarily affected Treg proliferation without affecting their survival.

### Example 4

### Jagged-1 and OX40L are Critical for GM-BMDC Mediated Treg-Expansion

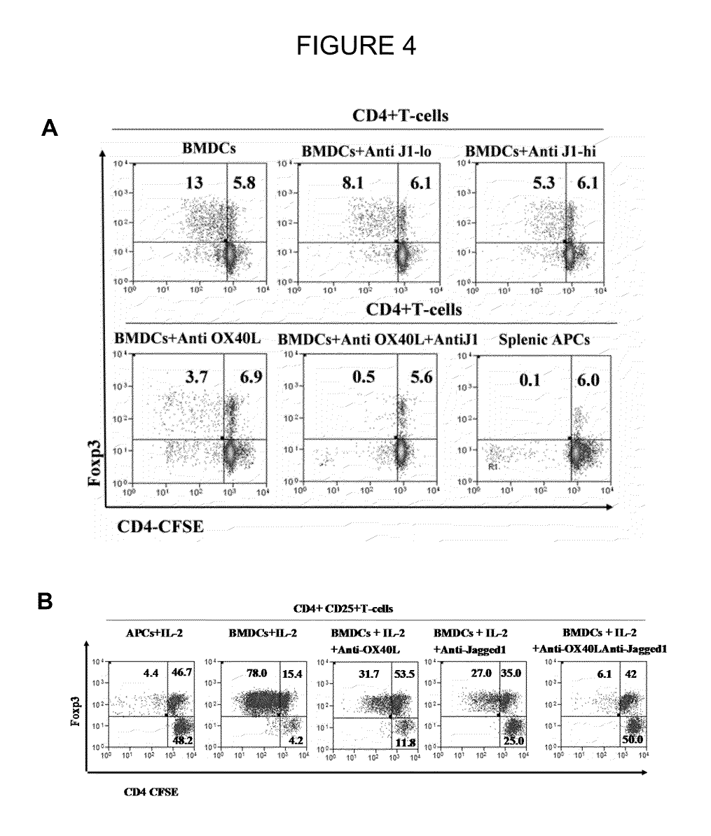

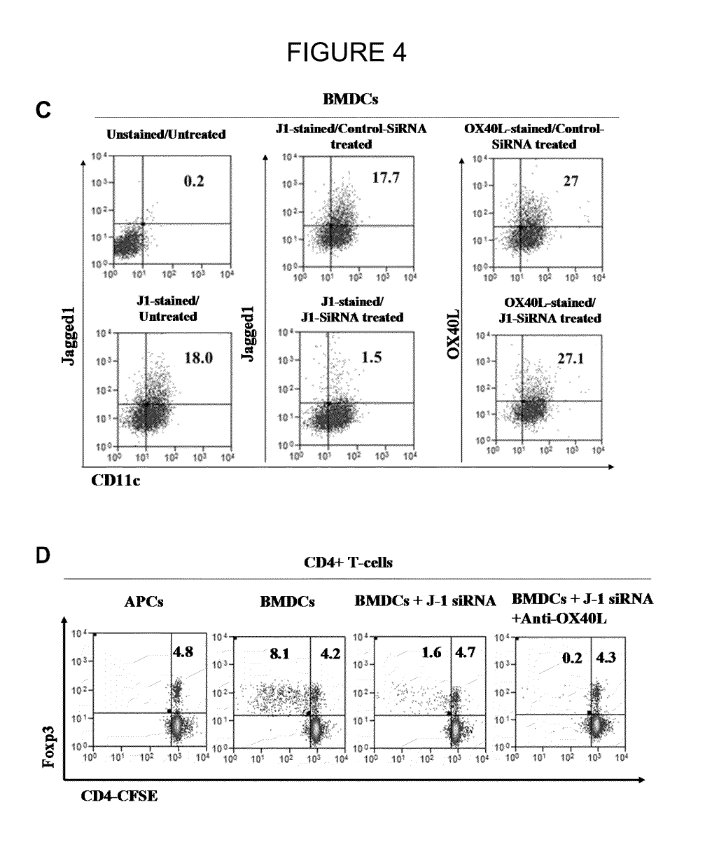

Specific antibodies to block OX40L and Jagged-1 were used to determine whether concurrent signaling by both ligands was essential for Treg expansion. Blocking either OX40L or Jagged-1, using specific antibodies, reduced Treg proliferation from 13.0% in the absence of antibody to 5.3±0.3% and 3.7±0.2% in the presence of anti-Jagged-1-Hi and anti-OX40L-Hi respectively. However, simultaneous blockade of both molecules completely prevented the GM-BMDC mediated Treg proliferation (13.0% v/s 0.5%±0.1%, p<0.01) (FIG. 4A). These data suggested that Notch signaling, likely induced by Jagged-1, along with OX40 signaling induced by OX40L were essential for GM-BMDC mediated Treg proliferation.

OX40L-mediated Treg expansion by GM-BMDC did not require TCR stimulation (8). To determine if the Jagged-1 mediated signaling was also independent of TCR signaling, CD25⁺ T cells were co-cultured with GM-BMDCs derived from MHC class-II^{-/-} mice in the presence of IL-2. MHC GM-BMDCs were able to expand Tregs (78.0±1.4%). However, blocking either OX40L or Jagged-1 significantly reduced Treg proliferation from 78.0±1.4% to 31.7±0.5% in the presence of anti-OX40L and to 27.0±1.1% in the presence of anti-Jagged-1. Blocking both ligands almost completely prevented Treg proliferation (p<0.01 in all instances) (FIG. 4B).

To further substantiate the relative importance of these two ligands, specific siRNA was used to knock down Jagged-1 (FIG. 4C) on GM-BMDC co-cultured with CD4+ Tcells. siRNA treatment (120 nM) significantly reduced expression of Jagged-1 in GM-BMDC (1.5±0.4%; p<0.01) relative to its expression on either untreated (18.0±2.1%) or control siRNA treated (17.7±2.4%) GM-BMDC, without altering expression of OX40L (approximately 27% in both Jagged-1 siRNA treated and control siRNA treated cells) (FIG. 4C, right panels). These GM-BMDCs were used in co-culture with CFSE-labelled naive CD4+ T-cells. Treg proliferation was significantly reduced from 8.1±1.0% in the presence of control GM-BMDC to 1.6±0.5% in the presence of Jagged-1 knocked down GM-BMDC (FIG. 4D). Combined treatment of GM-BMDC with an OX40L blocking antibody (hi=10 μg/ml) along with Jagged-1 inhibition almost completely abrogated their ability to expand Tregs (0.2±0.1%). These results clearly showed that both OX40L and Jagged-1 expressed on GM-BMDC were required for efficient Treg expansion.

### Example 5

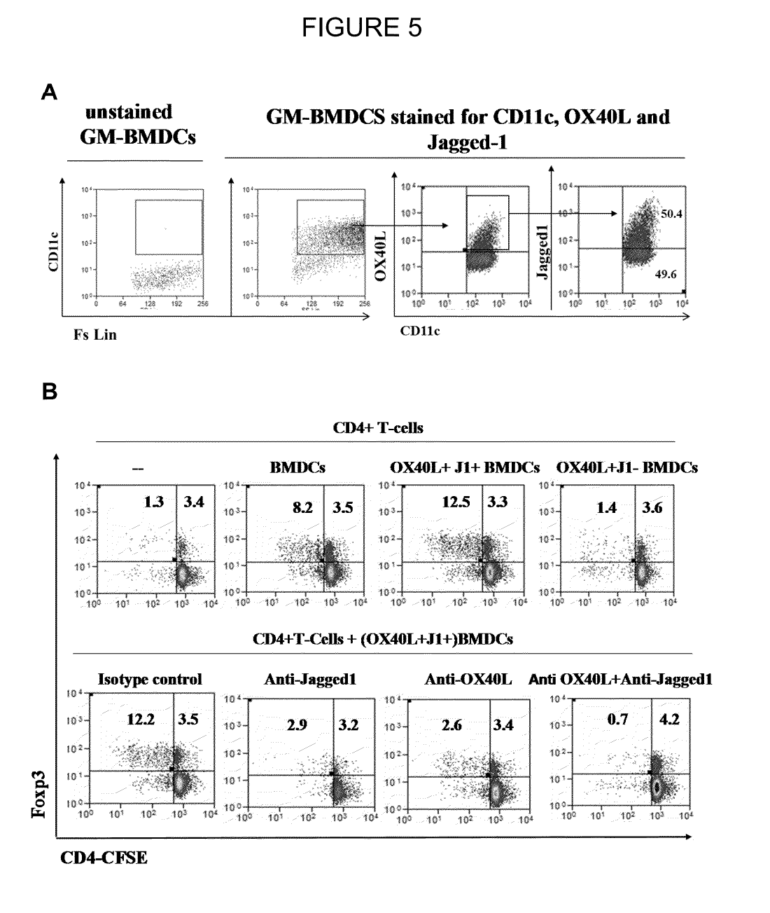

### OX40L and Jagged-1 Mediated Co-Signaling is Required for GM-BMDC Mediated Treg Expansion

[GM-BMDCs that were OX40L⁻ were also Jagged-1⁻ (FIG. 5A). On the other hand, about half of OX40L⁺ GM-BMDCs were Jagged-1⁺ (50.3±0.5%, p<0.02) (FIG. 5A).

To determine if OX40L and Jagged-1 co-expression was required for OX40L⁻ GM-BMDC-induced expansion of Tregs, the GM-BMDC were sorted into OX40L⁺ Jagged-1⁺ and OX40L⁺ Jagged-1⁻ DCs and used them in co-culture with naive CD4⁺ cells. While total GM-BMDC could induce Treg proliferation (e.g., 8.2%), the OX40L⁻ Jagged-1⁺ GM-BMDCs were able to more efficiently expand Tregs (12.5±0.2%). In contrast, OX40L⁺ Jagged-1⁻ failed to mediate significant expansion of Tregs (1.4±0.1%, p<0.001) (FIG. 5B). Blocking either ligand with the corresponding blocking antibody caused significant reduction in Treg expansion. However, blocking both ligands (anti-OX40L=10 g/ml, anti-Jagged-1=20 μg/ml) on OX40L⁺ Jagged-1^{|} GM-BMDCs abrogated Treg expansion (reduced from 12.5±0.2% to 0.7±0.1%; p<0.01). These results clearly demonstrated that GM-BMDC mediated ex vivo Treg expansion required cell surface expression of both OX40L and Jagged-1.

### Example 6

### GM-BMDC Associated Jagged-1 can Induce Treg Proliferation by Activating Treg Associated Notch3

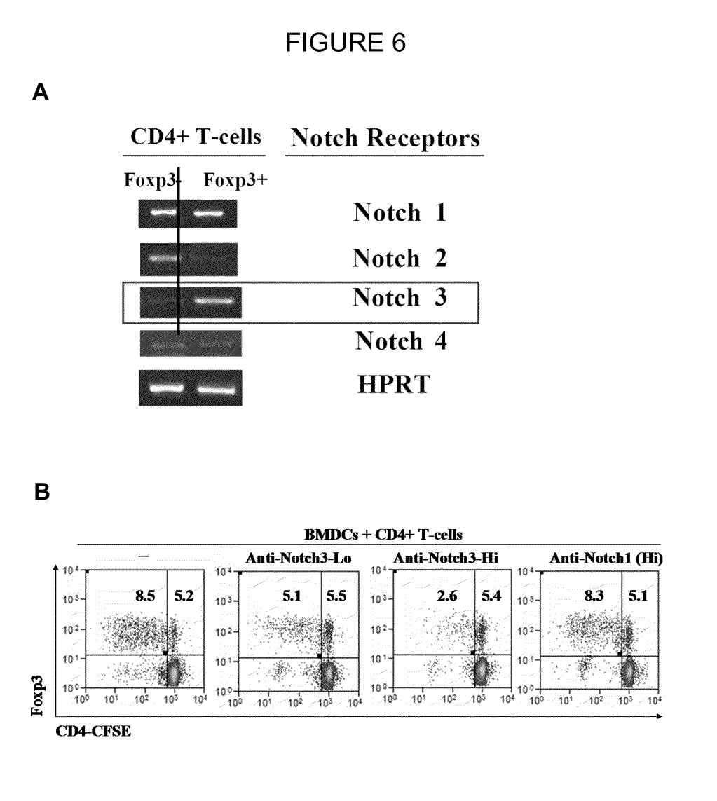

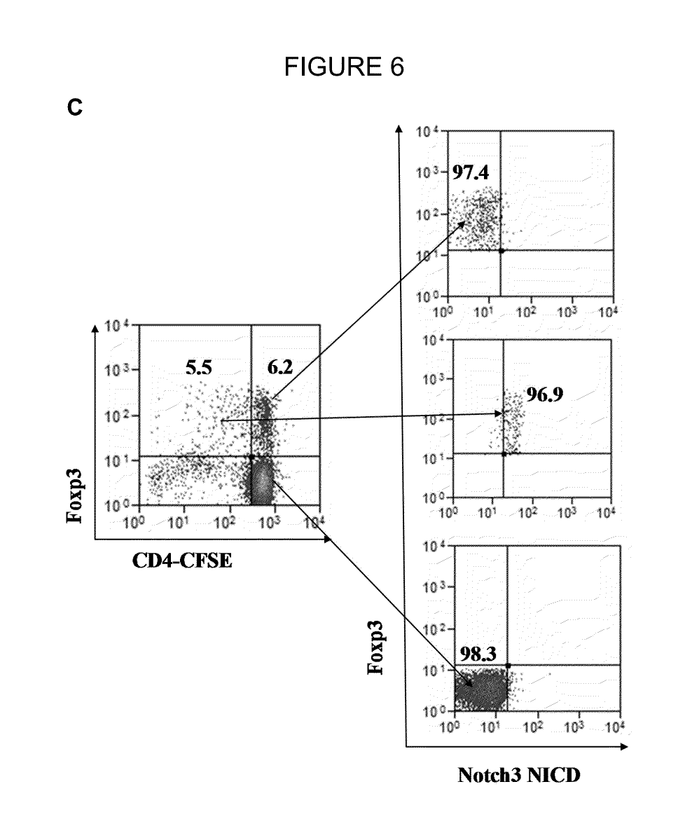

To determine the specific Notch receptor that was activated by Jagged-1 to cause Treg proliferation, mRNA expression patterns of all four Notch receptors in Foxp3⁺ (i.e. GFP⁺) and Foxp3⁻ (GFP⁻) cells from Foxp3-GFP mice were analyzed. Semi-quantitative PCR indicated that transcripts for Notch 1 and Notch 4 were similarly expressed in Teffs and Tregs. However, expression of Notch 3 transcript was significantly higher in Foxp3⁺ Tregs relative to Foxp3⁻ effector T cells, while the transcripts for Notch 2 was predominantly expressed in Teff cells (FIG. 6A). These findings suggested that Jagged-1 expressed on GM-BMDC may be binding specifically to Notch 3 expressed on Tregs to cause their expansion.

The importance of Notch 3 signaling was substantiated by a reduction in GM-BMDC induced Treg proliferation upon addition of a Notch 3 blocking antibody to the GM-BMDC-T cell co-culture in a dose dependent manner. The proliferation was reduced from 8.5±0.3% in untreated culture to 5.1±0.4% and 2.6±0.2% in the presence of low (10 μg) and high dose (20 μg) of anti-Notch3 antibody respectively: p<0.02 (FIG. 6B). In contrast, a blocking antibody to Notch 1 did not have any apparent effect on Treg proliferation.

Detection of cytoplasmic Notch Intra-Cellular Domain (NICD) has been used as a marker for activated Notch 3 (24). To confirm the role of Notch 3 in mediating Jagged-1 induced signaling, a Notch 3 specific polyclonal antibody (12, 21) was used to detect the intracellular portion of Notch 3 in the GM-BMDC/T-cell co-cultures. Analyses of proliferating and non-proliferating Foxp3⁺ and Foxp3⁻ cells showed that nearly 97% of the proliferating Foxp3⁺ T cells were positive for Notch 3 NICD, while approximately 98% of non-proliferating Foxp3⁺ or Foxp3⁻ T cells were negative for Notch 3 NICD (FIG. 6C). Collectively, the data suggested that Notch 3, expressed selectively on Tregs, is activated by Jagged-1 expressed on GM-BMDCs and this interaction is essential for Treg proliferation.

### Example 7

### OX40L⁺ Jagged-1⁻ GM-BMDCs can Suppress Ongoing EAT

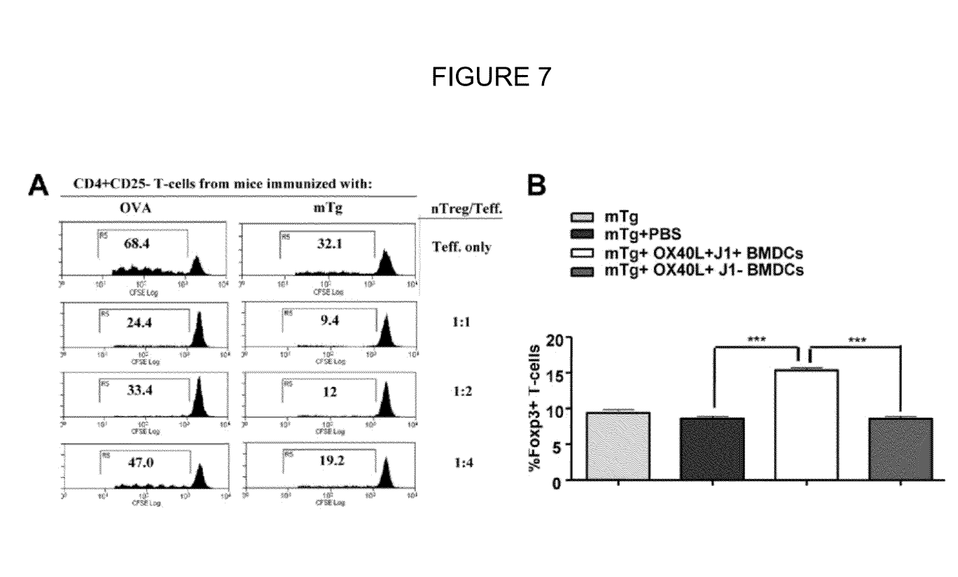

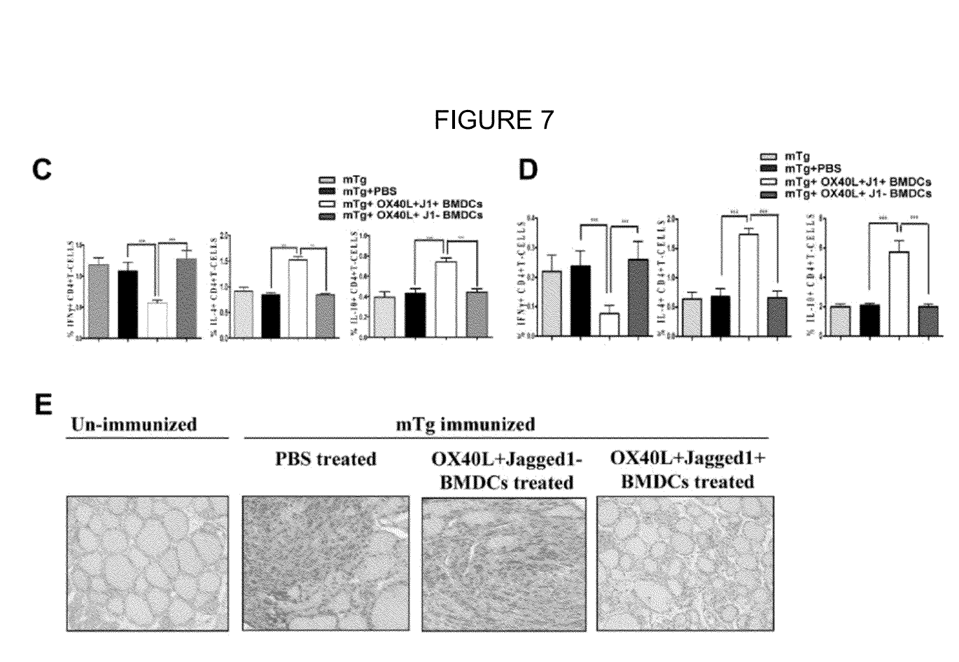

The suppressive effect of ex vivo generated Tregs on antigen-induced T cell proliferation was tested. Mice were immunized with 100 μg mTg or OVA to induce an antigen specific effector T cell response, which was monitored through the emergence of serum antibodies to mTg and OVA respectively. T cells from naïve mice were used to set up GM-BMDC/T-cell co-cultures to generate Tregs. In the absence of TCR stimulation, the expanded Tregs were a major fraction of the CD25⁺ T-cells and were therefore isolated on the basis of CD25 expression. CD4⁻CD25⁻ T cells were then isolated from the above-mentioned immunized animals, stained them with CFSE and co-cultured with splenic APCs in the presence of mTg or OVA with or without sorted Tregs (CD4⁺CD25⁺). CD25⁻ cells from OVA-immunized mice and mTg-immunized mice proliferated in the presence of OVA and mTg respectively. Exogenous OVA-induced proliferation was much more robust as compared to the autoantigen mTg-induced proliferation. Both mTg- and OVA-induced proliferations were significantly suppressed when CD25⁺ Tregs were added at either 1:1, 1:2, 1:4 Tregs:Teffs ratios (FIG. 7A). These results showed that ex vivo generated Tregs were functionally competent.

Since only a small fraction of GM-BMDC, viz. the OX40L⁺ Jagged-1⁺ fraction, could expand Tregs ex vivo, subpopulation of DCs were also tested to determine if they could also expand Tregs in vivo and confer protection against EAT. Mice were immunized with mTg+CFA on days 1 and 10 to induce EAT. On days 17 and 22, these mice were adoptively transferred with different subsets of GM-BMDC. Mice were sacrificed on day 35 and analyzed for Foxp3^{|} Tregs. The OX40L⁺ Jagged-1⁺ GM-BMDC recipient mice showed a significant increase in the percentage of Foxp3⁺ Tregs in the spleen (15.0±0.5%) compared to control mice that were treated with PBS (9.2±1.0%) or mice that received OX40L⁺Jagged-1⁻ GM-BMDC (9.0±0.5%) (p<0.01 v/s OX40L⁺Jagged-1⁺ GM-BMDC in both cases) (FIG. 7B). CD4⁺ T-cells from these recipient mice were re-stimulated with mTg in the presence of APCs for 3 days and analyzed for cytokine production. Mice that received OX40L⁺ Jagged-1⁺ GM-BMDC showed a significant decrease in IFNγ producing CD4⁺ T cells (p<0.01), while showing a significant increase (p<0.01) in IL-4⁺ and IL-10⁻ CD4⁺ T cells compared to controls (FIG. 7C). Similarly, the cytokine profile of T-cells from thyroid draining lymph nodes of OX40L Jagged-1^{|} GM-BMDC recipient mice showed significantly lower percentages of IFN-γ⁺ cells, while the percentages of IL-4⁺ and IL-10^{|} CD4^{|} T cells were significantly (p=0.001) higher (FIG. 7D) relative to the controls.

Thyroid histopathology revealed reduced infiltration of lymphocytes into the thyroid of OX40L⁺ Jagged-1⁺ GM-BMDC-recipient mice compared to the control groups either treated with OX40L^{|} Jagged-1⁻ GM-BMDCs or left untreated (p=0.02 in both cases; FIG. 7E). These results showed that OX40L⁺ Jagged-1⁺ GM-BMDC can increase the number of Tregs in vivo, with a concomitant decrease in Th1 cytokines and increase in suppressor cytokines, and suppress ongoing EAT. This observation is consistent with the earlier findings which showed that protection conferred by the treatment with low dose GM-CSF was primarily mediated through increased production of IL-10 as a result of expansion of IL10⁺CD4⁺Foxp3⁻ T-regs in these mice (6).

### Example 8

### Soluble OX40L & Jagged-1 Induce Treg Expansion and Prevent Diabetes Onset in NOD Mice

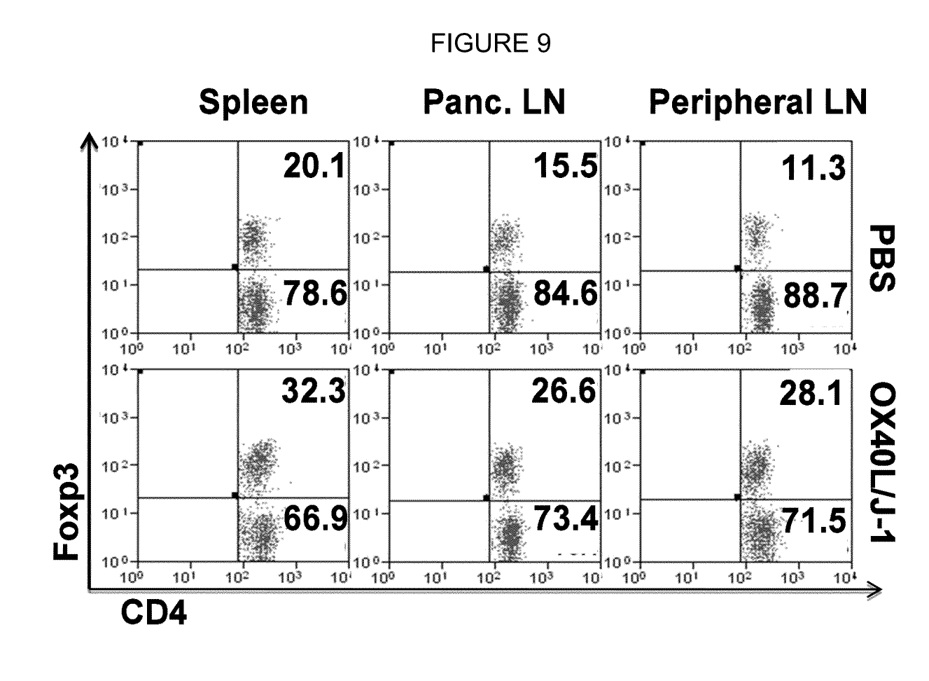

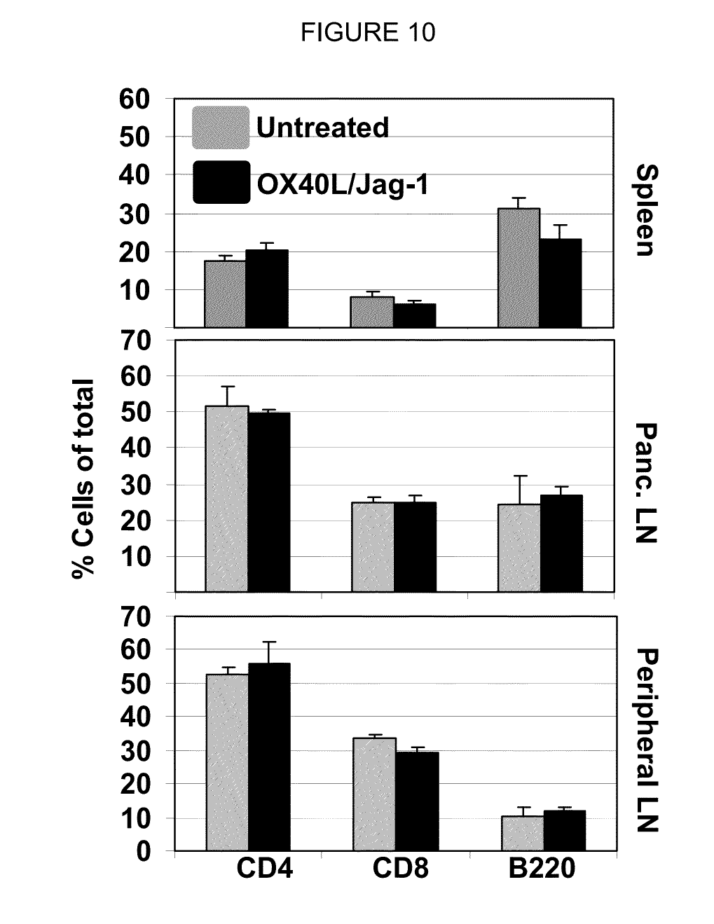

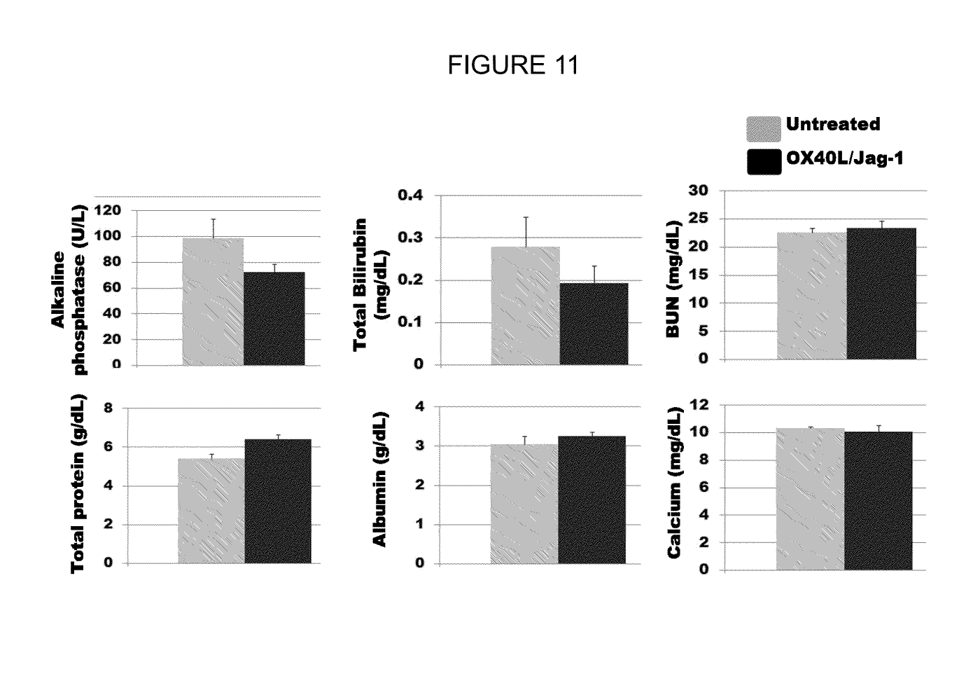

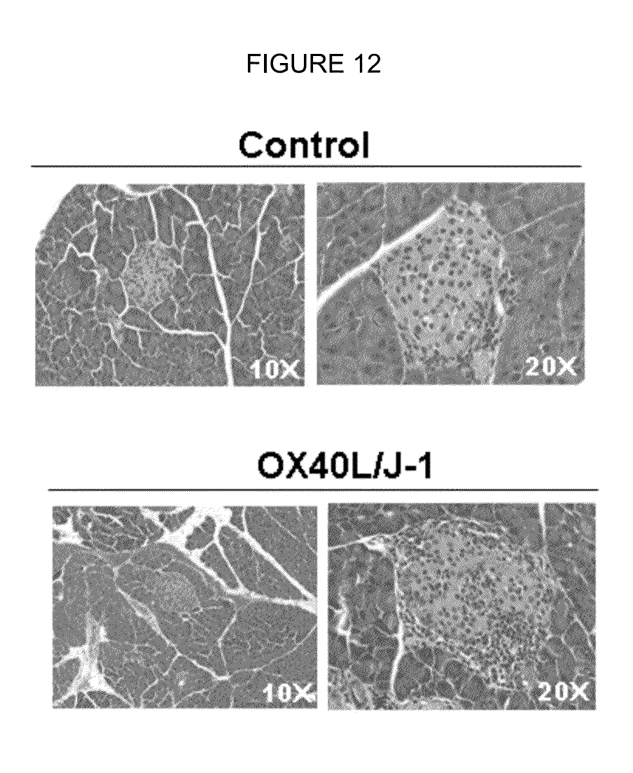

To investigate whether soluble OX40L- & Jagged-1-induced co-signaling can cause Treg expansion in vivo in non-obese diabetic (NOD) mice, a model of type 1 diabetes (T1D) was used. Ten-week old NOD mice were treated 3-times with PBS or soluble recombinant OX40L (200 μg/dose) and soluble recombinant Jagged-1 (100 μg/dose). Following treatment, mice were sacrificed and different tissues were analyzed for changes in the percentage of Tregs, CD4+ and CD8+ T lymphocytes and B cells. Mice receiving OX40L & Jagged-1 showed a significant increase in the percentage of Foxp3^{|} Tregs in the spleen (e.g., 20.1% in PBS treated vs 32.3% in ligand treated), pancreatic (15.5 vs 26.6%) and peripheral lymph nodes (11.3% vs 28.1%) (FIG. 9), indicating that soluble OX40L & Jagged-1 treatment can cause Treg expansion in vivo. Additionally, this treatment did not affect CD4⁺, CD8⁺ and B220⁺ cell numbers (FIG. 10) and did not alter the normal physiological function of the kidney and liver (FIG. 11). H&E staining of pancreatic tissues from treatment and control mice showed no β-cell damage upon OX40L/Jagged-1 treatment (FIG. 12). Mice receiving soluble OX40L and Jagged-1 remained diabetes-free for up to 15 weeks of age compared to control (100% in treatment group vs. 66.7%). This data suggested that treatment with OX40L and Jagged-1 caused a dramatic increase in Tregs and protected against onset of diabetes, without causing any adverse side effects.

### REFERENCES

1. Vasu et al., 2003, Selective induction of dendritic cells using granulocyte macrophage-colony stimulating factor, but not fms-like tyrosine kinase receptor 3-ligand, activates thyroglobulin-specific CD4+/CD25+ T cells and suppresses experimental autoimmune thyroiditis. J Immunol 170: 5511-22
2. Sheng et al., 2006. Suppression of experimental autoimmune myasthenia gravis by granulocyte-macrophage colony-stimulating factor is associated with an expansion of FoxP3+ regulatory T cells. J Immunol 177: 5296-306
3. Cheatem et al., 2009. Modulation of dendritic cells using granulocyte-macrophage colony-stimulating factor (GM-CSF) delays type 1 diabetes by enhancing CD4+CD25+ regulatory T cell function. Clin Immunol 131: 260-70
4. Gaudreau et al., 2007. Granulocyte-macrophage colony-stimulating factor prevents diabetes development in NOD mice by inducing tolerogenic dendritic cells that sustain the suppressive function of CD4+CD25+ regulatory T cells. J Immunol 179: 3638-47
5. Bernasconi et al., 2010. Granulocyte-macrophage colony-stimulating factor elicits bone marrow-derived cells that promote efficient colonic mucosa] healing. Inflamm Bowel Dis 16: 428-41
6. Ganesh et al., 2009. GM-CSF-induced CD11c+CD8a−-dendritic cells facilitate Foxp3+ and IL-10+ regulatory T cell expansion resulting in suppression of autoimmune thyroiditis. Int Immunol 21: 269-82
7. Gangi et al., 2005. IL-10-producing CD4+CD25+ regulatory T cells play a critical role in granulocyte-macrophage colony-stimulating factor-induced suppression of experimental autoimmune thyroiditis. J Immunol 174:7006-13
8. Bhattacharya et al., 2011. GM-CSF-induced, bone-marrow-derived dendritic cells can expand natural Tregs and induce adaptive Tregs by different mechanisms. Journal of leukocyte biology 89: 235-49
9. Godfrey et al., 1994. Identification of a human OX-40 ligand, a co-stimulator of CD4+ T cells with homology to tumor necrosis factor. J Exp Med 180: 757-62
10. Ruby et al., 2009. Cutting Edge: OX40 agonists can drive regulatory T cell expansion if the cytokine milieu is right. Journal of immunology 183: 4853-7
11. Griseri et al., 2010. OX40 is required for regulatory T cell-mediated control of colitis. The Journal of experimental medicine 207: 699-709
12. Kared et al., 2006. Jagged2-expressing hematopoietic progenitors promote regulatory T cell expansion in the periphery through notch signaling. Immunity 25: 823-34
13. Hoyne et al., 2000. Serrate1-induced notch signaling regulates the decision between immunity and tolerance made by peripheral CD4(+) T cells. Int Immunol 12: 177-85
14. Minter et al., 2005 Inhibitors of gamma-secretase block in vivo and in vitro T helper type 1 polarization by preventing Notch upregulation of Tbx21. Nat Immunol 6: 680-8
15. Fortini, 2009. Notch signaling: the core pathway and its posttranslational regulation. Dev Cell 16: 633-47
16. Amsen et al., 2004. Instruction of distinct CD4 T helper cell fates by different notch ligands on antigen-presenting cells. Cell 117: 515-26
17. Yvon et al., 2003. Overexpression of the Notch ligand, Jagged-1, induces alloantigen-specific human regulatory T cells. Blood 102: 3815-21
18. Vigouroux et al., 2003. Induction of antigen-specific regulatory T cells following overexpression of a Notch ligand by human B lymphocytes. J Virol 77: 10872-80
19. Elyaman et al., 2007. JAGGED-1 and delta 1 differentially regulate the outcome of experimental autoimmune encephalomyelitis. Journal of immunology 179: 5990-8
20. Bassil et al., 2011. Notch ligand delta-like 4 blockade alleviates experimental autoimmune encephalomyelitis by promoting regulatory T cell development. J Immunol 187: 2322-8
21. Lee et al., 2009. Arachidonic acid potentiates hypoxia-induced VEGF expression in mouse embryonic stem cells: involvement of Notch, Wnt, and HIF-1alpha. Am J Physiol Cell Physiol 297: C207-16
22. Esquivel et al., 1977. Induction of autoimmunity in good and poor responder mice with mouse thyroglobulin and lipopolysaccharide. J Exp Med 145:1250-63
23. Okamoto et al., 2009. Jagged-1 on dendritic cells and Notch on CD4+ T cells initiate lung allergic responsiveness by inducing IL-4 production. J Immunol 183: 2995-3003
24. Anastasi et al., 2003. Expression of activated Notch3 in transgenic mice enhances generation of T regulatory cells and protects against experimental autoimmune diabetes. J Immunol 171: 4504-11
25. Abbas et al., 1996. Functional diversity of helper T lymphocytes. Nature 383: 787-93
26. Campese et al., 2009. Notch3 and pTalpha/preTCR sustain the in vivo function of naturally occurring regulatory T cells. International immunology 21: 727-43
27. Vu et al., 2007. OX40 co-stimulation turns off Foxp3+ Tregs. Blood 110: 2501-10
28. Song et al., 2004. The co-stimulation-regulated duration of PKB activation controls T cell longevity. Nat Immunol 5: 150-8
29. Rogers et al., 2001. OX40 promotes Bcl-xL and Bcl-2 expression and is essential for long-term survival of CD4 T cells. Immunity 15: 445-55
30. So et al., 2011. OX40 complexes with phosphoinositide 3-kinase and protein kinase B (PKB) to augment TCR-dependent PKB signaling. Journal of immunology 186: 3547-55
31. Vacca et al., 2006. Notch3 and pre-TCR interaction unveils distinct NF-kappaB pathways in T-cell development and leukemia. EMBO J 25: 1000-8
32. Barbarulo et al., 2011. Notch3 and canonical NF-kappaB signaling pathways cooperatively regulate Foxp3 transcription. J Immunol 186: 6199-206
33. Samon et al., 2008. Notch1 and TGF-beta 1 cooperatively regulate Foxp3 expression and the maintenance of peripheral regulatory T cells. Blood 112: 1813-21
34. Shevach et al., 2006. The lifestyle of naturally occurring CD4+ CD25+ Foxp3+ regulatory T cells. Immunological reviews 212: 60-73
35. Allan et al., 2005. The role of 2 FOXP3 isoforms in the generation of human CD4+ Tregs. The Journal of clinical investigation 115: 3276-84
36. Sakaguchi, 2004. Naturally arising CD4+ regulatory t cells for immunologic self-tolerance and negative control of immune responses. Annu Rev Immunol 22: 531-62
37. Sakaguchi et al., 1985. Organ-specific autoimmune diseases induced in mice by elimination of T cell subset. I. Evidence for the active participation of T cells in natural self-tolerance; deficit of a T cell subset as a possible cause of autoimmune disease. J Exp Med 161: 72-87
38. Zwar et al., 2006. CD4+CD25+ regulatory T cells inhibit the antigen-dependent expansion of self-reactive T cells in vivo. J Immunol 176: 1609-17
39. Tang et al., 2004. In vitro-expanded antigen-specific regulatory T cells suppress autoimmune diabetes. J Exp Med 199: 1455-65
40. Joetham et al., 2009. Antigen specificity is not required for modulation of lung allergic responses by naturally occurring regulatory T cells. J Immunol 183: 1821-7

Various changes and modifications to the disclosed embodiments will be apparent to those skilled in the art. Such changes and modifications may be made without departing from the spirit and scope thereof.

1. A method of expanding T-regulatory cells comprising co-culturing said T-regulatory cells with one or more of a OX40L^{|} bone marrow derived dendritic cell culture differentiated in the presence of GM-CSF, a Jagged-1⁺ bone marrow derived dendritic cell culture differentiated in the presence of GM-CSF and a OX40L⁺Jagged-1⁻ bone marrow derived dendritic cell culture differentiated in the presence of GM-CSF.

2. A method of treating an autoimmune disease in a patient in need of such treatment comprising administering to the patient a therapeutically effective amount of T-regulatory cells prepared according to the method of claim 1.

3. A method for expanding T-regulatory cells comprising co-culturing said T-regulatory cells with one or more of soluble OX40L and soluble Jagged-1.

4. The method of claim 3 wherein the OX40L and Jagged-1 are recombinantly produced.

5. A method of treating an autoimmune disease in a patient in need of such treatment comprising administering to the patient a therapeutically effective amount of one or more of soluble OX40L and soluble Jagged-1.

6. The method of claim 5 wherein the autoimmune disease is an autoimmune thyroid disease.

7. The method of claim 6 wherein the autoimmune thyroid disease is Grave's disease or Hashimoto disease.

8. The method of claim 5 wherein the autoimmune disease is Type 1 Diabetes mellitus.

9. The method of claim 5 wherein said OX40L and Jagged-1 are recombinantly produced.

10. The method of claim 5 wherein said patient is a human patient.

11. A method of treating an autoimmune disease in a patient in need of such treatment comprising administering to the patient a therapeutically effective amount of one or more of OX40L⁺ bone marrow derived dendritic cells differentiated in the presence of GM-CSF, Jagged-1⁻ bone marrow derived dendritic cells differentiated in the presence of GM-CSF and OX40L⁺Jagged-1⁺ bone marrow derived dendritic cells differentiated in the presence of GM-CSF.

12. The method of claim 11 wherein the autoimmune disease is an autoimmune thyroid disease.

13. The method of claim 12 wherein the autoimmune thyroid disease is Grave's disease or Hashimoto disease.

14. The method of claim 11 wherein the autoimmune disease is Type 1 Diabetes mellitus.

15. The method of claim 11 wherein said OX40L and Jagged-1 are recombinantly produced.

16. The method of claim 11 wherein said patient is a human patient.
